(19) United States
(12) Patent Application Publication
(10) Pub. No.: US 2012/0316106 A1
(43) Pub. Date: Dec. 13, 2012

(54) SYNERGISTIC ANTIBIOTIC COMBINATIONS AND DERIVATIVES

(75) Inventors: Ada Yonath, Rehovot (IL); Chen Davidovich, Rehovot (IL); Ella Zimmerman, Rehovot (IL); Anat Bashan, Rehovot (IL); Tamar Auerbach, Rehovot (IL); Matthew Belousoff, Rehovot (IL); Liqun Xiong, Chicago, IL (US); Dorota Klepacki, Chicago, IL (US); Alexander S. Mankin, River Forest, IL (US)

(73) Assignee: YEDA RESEARCH AND DEVELOPMENT CO. LTD., Rehovot (IL)

(21) Appl. No.: 13/519,862

(22) PCT Filed: Dec. 29, 2010
(86) PCT No.: PCT/IL2010/001089

§ 371 Date: Jun. 28, 2012

Related U.S. Application Data
(60) Provisional application No. 61/290,555, filed on Dec. 29, 2009.

(51) Int. Cl. A61K 31/7048; A61K 31/424; A61K 38/12; A61P 31/04; A61K 31/165; A61K 31/7056; C07H 17/08; A61K 31/366; A61K 31/215

(52) U.S. Cl. 514/2.9; 514/456; 514/29; 514/375; 514/2.3; 514/511; 514/628; 514/28; 514/24; 536/16.8

(57) ABSTRACT

Improved antibiotic analogs, and synergistic combinations of antibiotics designed based on structural crystallographic analysis, are provided as well as pharmaceutical compositions that include these improved analogs and synergistic combinations, along with methods for their production and use. The synergistic combinations target neighboring sites in the ribosome demonstrating the importance of the corresponding ribosomal sites for development of clinically-relevant synergistic antibiotics.

### FIELD OF THE INVENTION

The present invention relates to improved antibiotic derivatives, to synergistic antibiotic combinations, to compositions comprising them and to chemical tools for structure based drug design for identifying synergistic antibiotic combinations and antibiotic improvements.

### BACKGROUND OF THE INVENTION

Biochemical, genetic and functional evidence indicate that a great variety of antibiotics inhibit protein synthesis by binding to ribosomal functional regions. The ribosome is a bio-macromolecular multi-component assembly that is universal to all forms of known life. It translates the genetic code and produces proteins. It consists of two unequally sized subunits that act together in protein biosynthesis. Decoding and mRNA transit take place on the small subunit, while the large subunit provides the machinery for peptide bond formation, nascent protein chain elongation and its protection.

While ribosomal interfering antibiotics have been in clinical use since the 1950's, rapid resistance, cross-resistance and drug toxicity drive the need for new treatments for bacterial infection.

Crystallographic studies, performed over the last decade, revealed the exact binding sites of a variety of antibiotics (Poehlsgaard, J. and Douthwaite, S. 2005, Nat Rev Microbiol 3, 870-881). Many natural antibiotics, as well as their clinically relevant semisynthetic derivatives, bind at the peptidyl transferase center (PTC) in the large ribosomal subunit (50S). The PTC provides binding pockets for phenicols (e.g. chloramphenicol), lincosamides (e.g. clindamycin), pleuromutilins (e.g. tiamulin, retapamulin) and oxazolidinones (e.g. linezolid) (Hansen, J. L. et al., 2003, J Mol Biol 330, 1061-1075; Schluenzen, F. et al. 2004, Mol Microbiol 54, 1287-1294; Harms, J. et al., 2004, BMC Biol 2, 4,1-10; Tu, D. et al., 2005, Cell 121, 257-270; Leach, K. L. et al., 2007, Mol Cell 26, 393-402; Davidovich, C. et al., 2007, Proc Natl Acad Sci USA 104, 4291-4296; Ippolito, J. A. et al., 2008, J Med Chem 51, 3353-3356; Gurel, G. et al., 2009, J Mol Biol 389, 146-156; Auerbach, T. et al., 2009, Biotechnolog 84, 24-35). Most of these compounds inhibit cell growth by interfering with peptide bond formation (Polacek, N. and Mankin, A. S., 2005, Crit Rev Biochem Mol Biol 40, 285-311). An additional major antibiotic binding site in the large ribosomal subunit is located at the upper segment of the nascent protein exit tunnel (NPET), which is adjacent to the PTC, and is used by macrolides and type B streptogramins (Schluenzen, F. et al., 2003, Structure (Camb) 11, 329-338, Berisio, R. et al., 2003, Nat Struct Biol 10, 366-370). Binding to this site seems to impede progression of the nascent proteins towards the tunnel exit. Thus, compounds binding to the PTC and NPET inhibit successive steps in protein synthesis: formation of the nascent chains and their export from the ribosome.

A potential way to overcome some of the issues that plague antibiotic treatment is to use pairs of antibiotics that inhibit the ribosome at two different positions. Simultaneous inhibition of successive steps of a specific biochemical pathway often results in a synergistic action of the inhibitors (Acar, J. F., 2000, Med Clin North Am 84, 1391-1406). Nature has not ignored this opportunity when evolving ribosomal antibiotics. For example, streptogramin antibiotics, produced by several Streptomyces species, are secreted as a combination of two structurally distinct compounds that inhibit cell growth by acting upon the PTC and NPET (Cocito, C. et al.,1997, J Antimicrob Chemother 39 (Suppl A) . Streptogramin A (S_{A}) compounds are cyclic poly-unsaturated macrolactones that bind in the PTC, whereas type B streptogramins (S_{B}) compounds are cyclic depsipeptides that bind in the NPET. Each of the individual streptogramin components is a fairly weak antibiotic on its own, but in combination they exhibit strong inhibitory effect. One synergistic ribosomal antibiotic has already found use in the clinic. Synercid™, a pair of semisynthetic streptogramins (Quinupristin/Dalfopristin) is currently used as a synergistic pair of drugs against gram-positive bacteria in the clinic as a last resort treatment for methicillin-resistant Staphylococcus aureus (MRSA) and vancomycin-resistant Enterococcus faecium (VREF) (Manfredi R & Sabbatani S,2010, Braz J Infect Dis 14:96-108). Since its clinical approval by the FDA in 1999 this drug combination suffers very little resistance by MRSA (Baudoux P, et al., 2010, J Antimicrob Chemother 65:1228-1236). Synergistic behavior of the streptogramins is driven by the streptogramin A (Dalfopristin) member, which upon binding to the 50S subunit significantly increases the Kₐ of the streptogramin B (Quinupristin) component.

Lankacidin C (LC) and lankamycin (LM) are two inhibitory compounds produced by Streptomyces rochei 7434AN4 (Kinashi, H. et al., 1994, J Antibiot (Tokyo) 47, 1447-1455, Arakawa et al., 2005, Chem. Biol. 12:249-256; Suzuki et al., 2010, Biosci. Biotechnol. Biochem. 74:819-827). The structures of LC and LM are chemically distinct and rather different from those of streptogramins (FIG. 1). LC is a macrocyclic compound composed of a 17-membered carbocyclic ring, bridged by a 6-membered lactone. LM is a macrolide whose 14-member lactone ring is decorated with 4-acetyl-L-arcanose and D-chalcose sugars resembling erythromycin (ERY) (FIG. 1). LC, which is used in veterinary medicine, inhibits growth of Gram-positive bacteria by interfering with protein synthesis but has little effect on eukaryotic cell-free translation. LC and its derivatives also exhibit antitumor activity although it is unclear whether this effect is related to the drug's effect upon protein synthesis. Lankamycin exhibits a weak antibiotic activity against several Gram-positive bacteria and low toxicity in animal models.

LC competition with chloramphenicol for binding to the ribosome reveals the large ribosomal subunit as a likely target of its action, in accord with its classification as a protein synthesis inhibitor. Co-regulation of production of LC and LM (Arakawa, K. et al., 2007, Microbiology 153 (Pt 6), 1817-1827) suggests that these drugs have been evolutionarily optimized to work together. Nevertheless, although LC and LM are co-produced by the Streptomyces strain, there has been no information about their sites of action, nor any evidence of functional interaction between these two antibiotic compounds.

Arakawa et al. (Antimicrobial agents and chemotherapy 2006, 60, 1946-1952) describes deoxylankamycin molecules (at C-15 and C-8 positions) isolated from mutant bacteria having decreased antimicrobial activity in the reverse order of the number of hydroxyl groups, and (Actinomycetologica 2008, 22:35-41) additional mutant lankamycine molecules having no antimicrobial activities.

WO 03/026562 to some of the inventors of the present application discloses methods for growing crystals of free and antibiotic-complexed large ribosomal subunits, suitable for crystallographic analysis and methods for utilizing the coordinates obtained for rational design or identification of antibiotics.

US 2009/0042186 to Mankin et al. discloses methods for mapping and identifying new sites for antibiotic action in the ribosome of a microorganism using a random mutant library of the ribosomal RNA genes of the microorganism.

U.S. Pat. No. 7,606,670 to Ramakrishnan et al. claims a method of identifying an inhibitor of a small (30S) ribosomal subunit using x-ray atomic coordinates to identify an active site.

U.S. Pat. No. 7,169,756 to Koteva et al. discloses cyclic streptogramin type B peptide antibiotics comprising the replacement of an enzyme sensitive ester bond with a stable bond selected from an amide, N-methyl amide, enamine or sulfonamide bond.

McFarland et al. (Antimicrobial Agents and Chemotherapy 1984, 25, 226-233), describe Lankacidin derivatives containing side chain modifications of Lankacidin group antibiotics.

Auerbach T, et al. (Proceedings of the National Academy of Sciences of the United States of America 2010, 107:1983-1988) describe the structure of ribosome-lankacidin complex revealed by crystallographic analysis.

There remains an unmet need for improved antibiotics and antibiotic combinations having synergistic activity and principles for their structure-based design.

### SUMMARY OF THE INVENTION

The present invention provides improved antibiotic derivatives, synergistic combinations and pharmaceutical compositions of antibiotics based on structural crystallographic analysis.

The antibiotic derivatives of the present invention and the synergistic combinations are designed to possess improved binding to the large ribosomal subunit and/or to permit the binding of a second antibiotic molecule to another binding site on the large ribosomal subunit.

According to one aspect, the present invention provides a synergistic combination of antibiotics comprising two antibiotic molecules capable of binding simultaneously at neighboring sites of the ribosomal large subunit, wherein one antibiotic is capable binding to the peptidyl transferase center (PTC) site of the ribosomal large subunit and one antibiotic is capable of binding to the nascent peptide exit tunnel (NPET) site of the same ribosomal large subunit.

According to some embodiments of this aspect a synergistic combination is provided comprising two antibiotic molecules capable of binding simultaneously to neighboring sites of a ribosomal large subunit, wherein one antibiotic is capable binding to the peptidyl transferase center (PTC) site of the ribosomal large subunit and the other antibiotic is capable of binding to the nascent peptide exit tunnel (NPET) site of the ribosomal large subunit, wherein at least one of said antibiotic molecules is selected from the group consisting of Lankacidin C (LC), Lankamycin (LM), Virginiamycin M, Virginiamycin S, Pleuromutilin, Chloramphenicol, Methylmycin and Clindamycin, and wherein the two antibiotic molecules provide a synergistic anti-bacterial effect. Each possibility represents a separate embodiment of the invention.

According to some embodiments the synergistic combination comprises isolated Lankacidin C (LC).

According to other embodiments the synergistic combination comprises Lankamycin (LM) or a derivative thereof. Each possibility represents a separate embodiment of the invention.

According to some embodiments the synergistic combination comprises a deoxy LM derivative represented by the structure of Formula I:

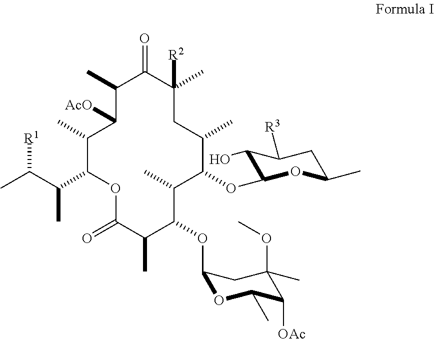

wherein: R¹ is OH, R² is H, and R³ is OMe; or

R¹ is H, R² is OH, and R³ is OMe; or
R¹ is H, R² is H, and R³ is OMe.

According to other embodiments, the synergistic combination comprises a desoamine derivative according to Formula I wherein: R¹ is OH, R² is OH, and R³ is selected from the group consisting of: NMe₂, NHMe and NH₂. Each possibility represents a separate embodiment of the invention.

According to yet additional embodiments, the synergistic combination comprises a C-15 LM derivative according to Formula I wherein: R¹ is an amine-containing group, R² is OH, and R³ is OMe or NMe₂; According to some specific embodiments R¹ is selected from the group consisting of: amine, N-methyl amine, N-dimethyl amine and N-ethyl amine. Each possibility represents a separate embodiment of the invention.

According to yet other embodiments, the LM derivative is 3-O-arcanosyl LM represented by the structure of Formula II:

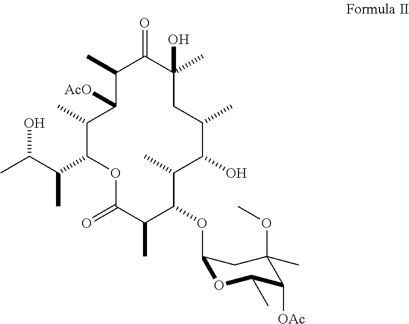

According to some embodiments, the synergistic combination comprises Lankamycin (LM) or a derivative thereof and a type A Streptogramin (S_{A}). Each possibility represents a separate embodiment of the invention.

According to other embodiments, the synergistic combination comprises Lankacidin C (LC) or a derivative thereof and a type B Streptogramin (S_{B}).

According to yet other embodiments the synergistic combination comprises one type A Streptogramin (S_{A}), and one type B Streptogramin (S_{B}).Explicit ly excluded from the scope of the present invention is the known synergistic pair of antibiotics Dalfopristin and Quinupristin. Each possibility represents a separate embodiment of the invention.

According to yet another aspect, the present invention provides pharmaceutical compositions comprising two isolated antibiotic molecules capable of binding simultaneously to neighboring sites of a ribosomal large subunit, wherein one antibiotic is capable binding to the peptidyl transferase center (PTC) site of the ribosomal large subunit and the other antibiotic is capable of binding to the nascent peptide exit tunnel (NPET) site of the ribosomal large subunit, wherein at least one of said isolated antibiotic molecules is selected from the group consisting of Lankacidin C (LC), Lankamycin (LM), Virginiamycin M, Virginiamycin S, Pleuromutilin, Chloramphenicol, Methylmycin and Clindamycin, and wherein the two antibiotic molecules provide a synergistic anti-bacterial effect. Each possibility represents a separate embodiment of the invention.

According to some embodiments the pharmaceutical composition comprises at least one PTC binding component selected from the group consisting of: Lankacidin C (LC), LC derivative, Dalfopristin and Virginiamycin M. According to other embodiments the PTC binding component is selected from: phenicols, lincosamides, pleuromutilins and oxazolidinones. According to yet other embodiments the PTC binding component is selected from the group consisting of: Chloramphenicol, Clindamycin, Tiamulin, Retapamulin and Linezolid. Each possibility represents a separate embodiment of the invention.

According to other embodiments pharmaceutical compositions comprise at least one NPET bonding component selected from the group consisting of: Lankamycin M (LM), LM derivative, Quinupristin, and Virginiamycin S. Each possibility represents a separate embodiment of the invention. Explicitly excluded from the scope of the present invention is the known synergistic pair of antibiotics Dalfopristin and Quinupristin.

According to some embodiments the pharmaceutical composition comprises Lankacidin C (LC), and a macrolide antibiotic. According to other embodiments the pharmaceutical composition comprises Lankacidin C (LC) and a ketolide antibiotic. Each possibility represents a separate embodiment of the invention. According to some embodiments the pharmaceutical composition comprises

Lankacidin C (LC) and a type B Streptogramin (S_{B}) antibiotic. Each possibility represents a separate embodiment of the invention.

According to yet other embodiments, the pharmaceutical composition comprises Lankamycin M (LM) or a derivative thereof, and a type A Streptogramin (S_{A}) antibiotic. Each possibility represents a separate embodiment of the invention.

According to yet other embodiments, the LM derivative is selected from the group consisting of: deoxy-LM derivative, desoamine-LM derivative, C-15 LM derivative and 3-O-arcanosyl LM.

According to some specific embodiments, the present invention provides a pharmaceutical composition comprising isolated Lankacidin C (LC), or an improved derivative thereof, and at least one antibiotic selected from the group consisting of: Lankamycin (LM), Quinupristin, and Virginiamycin S or their derivatives. Each possibility represents a separate embodiment of the invention.

According to other embodiments, the present invention provides a pharmaceutical composition comprising isolated Lankamycin M (LM), or an improved derivative thereof, and at least one antibiotic selected from the group consisting of: Lankacidin C (LC), Dalfopristin, Virginiamycin M, Pleuromutilin, Chloramphenicol and Methylmycin. Each possibility represents a separate embodiment of the invention.

According to yet other embodiments, the pharmaceutical composition comprises a synergistic combination of two antibiotics, wherein the synergistic combination is selected from the group consisting of: isolated Lankacidin C (LC) and isolated Lankamycin (LM); Lankacidin C (LC) and Quinupristin; Lankacidin C (LC) and Virginiamycin S; Lankamycin (LM) and Dalfopristin; Lankamycin (LM) and Virginiamycin M; Dalfopristin and Virginiamycin S; and Quinupristin and Virginiamycin M. Each possibility represents a separate embodiment of the invention.

According to yet other embodiments, the pharmaceutical composition comprises a synergistic combination of two antibiotic derivatives, wherein the synergistic combination is selected from the group consisting of: Lankacidin C (LC) and Lankamycin (LM) derivatives;

Lankacidin C (LC) and Quinupristin derivatives; Lankacidin C (LC) and Virginiamycin S derivatives; Lankamycin (LM) and Dalfopristin derivatives; Lankamycin (LM) and Virginiamycin M derivatives; Dalfopristin and Virginiamycin S derivatives; and Quinupristin and Virginiamycin M derivatives. Each possibility represents a separate embodiment of the invention.

According to some particular embodiments, the pharmaceutical composition comprises isolated Lankacidin C (LC) and isolated Lankamycin (LM).

According to yet other embodiments the pharmaceutical composition comprises Lankacidin C (LC) and a derivative of Lankamycin (LM). Each possibility represents a separate embodiment of the invention.

According to yet other embodiments the LM derivative is selected from the group consisting of: deoxy-LM derivative, desoamine-LM derivative, C-15 LM derivative and 3-O-arcanosyl LM. Each possibility represents a separate embodiment of the invention.

According to yet other embodiments, the LM derivative is according to Formulae I or II as defined above. Each possibility represents a separate embodiment of the invention.

According to another aspect, the present invention provides a chimeric molecule comprising two portions of two different antibiotic molecules capable of binding simultaneously at neighboring sites of a bacterial ribosomal large subunit.

According to some embodiments the chimeric molecule comprises: i. at least a portion of a molecule capable of binding to the peptidyl transferase center (PTC) site of the ribosomal large subunit; and ii. at least a portion of a molecule capable of binding to the nascent peptide exit tunnel (NPET) site of the same ribosomal large subunit; wherein (i) and (ii) bind simultaneously to the ribosomal large unit.

According to some embodiments, the chimeric molecule comprises at least a portion of Lankacidin C (LC) and at least a portion of Lankamycin M (LM). Each possibility represents a separate embodiment of the invention.

According to some embodiments, the chimeric molecule comprises at least one portion of Lankadicin C, and at least one portion of a type B Streptogramin (S_{B}) molecule. Each possibility represents a separate embodiment of the invention.

According to other embodiments, the chimeric molecule comprises at least one portion of Lankamycin, and at least one portion of a type A Streptogramin (S_{A}) molecule. Each possibility represents a separate embodiment of the invention.

According to other embodiments, the chimeric molecule comprises at least one portion of a type A Streptogramin (S_{A}) molecule, and at least one portion of a type B Streptogramin (S_{B}) molecule. According to another aspect, the present invention provides a covalently conjugated molecule comprising at least two antibiotic molecules capable of binding simultaneously at neighboring sites of ribosomal large subunit.

According to some embodiments the covalently conjugated molecule comprises one molecule capable of binding to the peptidyl transferase center (PTC) site of the ribosomal large subunit and one molecule capable of binding simultaneously to the nascent peptide exit tunnel (NPET) site of the same ribosomal large subunit.

According to some embodiments, the conjugated molecule comprises Lankacidin C (LC) and Lankamycin M (LM) or a derivative thereof. Each possibility represents a separate embodiment of the invention.

According to some embodiments, the conjugated molecule comprises Lankadicin C, and a type B Streptogramin (S_{B}) molecule. Each possibility represents a separate embodiment of the invention.

According to other embodiments, the conjugated molecule comprises Lankamycin or a derivative thereof, and a type A Streptogramin (S_{A}) molecule. Each possibility represents a separate embodiment of the invention.

According to yet other embodiments, the conjugated molecule comprises at least one type A Streptogramin (S_{A}) molecule, and at least one type B Streptogramin (S_{B}) molecule. Each possibility represents a separate embodiment of the invention.

According to some embodiments, the type A Streptogramin (S_{A}) molecule is selected from Dalfopristin and Virginiamycin M and the type B Streptogramin (S_{B}) molecule is selected from Quinupristin and Virginiamycin S. Each possibility represents a separate embodiment of the invention.

According to another aspect, the present invention provides derivatives of antibiotics designed in accordance with the binding sites identified using structural crystallographic analysis, having at least one improved property compared to the parent antibiotic molecule.

According to some embodiments, the improved property is selected from the group consisting of: enhanced biological activity, reduced toxicity, enhanced permeability, enhanced bioavailability, and reduced bacterial resistance.

According to some embodiments the unmodified parent antibiotic molecule is a macrolide. According to some specific embodiments the macrolide is Lankamycin (LM). According to other embodiments the parent antibiotic molecule is Lankacidin C (LC).

According to other embodiments the unmodified parent molecule is a Streptogramin antibiotic.

According to specific embodiments the Streptogramin antibiotic is a type A Streptogramin (S_{A}).

According to some embodiments, a macrolide derivative comprising a positively charged moiety is provided. According to some embodiments, the positively charged moiety is part of a sugar moiety. According to some specific embodiments the positively charged sugar moiety is desoamine. According to some embodiments, the macrolide derivative is a lankamycin (LM) derivative. According to some embodiments the lankamycin derivative comprises a positively charged desoamine pendant sugar moiety.

According to other embodiments, an improved antibiotic derivative is provided comprising at least one modification selected from the group consisting of: i. physical enlargement of the parent molecule; ii. covalent linkage of the two antibiotic molecules; and iii. modification of the parent molecule to further strengthen its binding to the ribosome.

According to some embodiments modification of the parent molecule to further strengthen its binding comprises adding a positively charge moiety. According to some specific embodiments an LM molecule is modified by adding or replacing existing moiety with a positively charged desoamine pendant sugar moiety.

According to some specific embodiments a desoamine LM derivative represented by the structure of Formula I is provided:

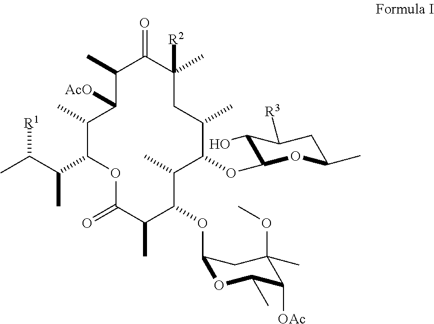

wherein: R₁ is OH, R₂ is OH, and R₃ is selected from the group consisting of: NMe₂, NHMe, and NH₂. Each possibility represents a separate embodiment of the invention.

According to yet additional embodiments, a LM derivative according to Formula I is provided wherein: R₁ is an amine-containing group, R₂ is OH, and R₃ is OMe or NMe₂; According to some specific embodiments R₁ is selected from the group consisting of: amine, N-methyl amine, N-dimethyl amine and N-ethyl amine. Each possibility represents a separate embodiment of the invention.

According to yet other embodiments, the LM derivative is 3-O-arcanosyl LM represented by the structure of Formula II:

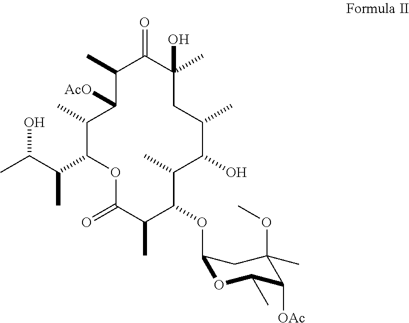

According to other embodiments the Streptogramin antibiotic is a type B Streptogramin (S_{B}).

Use of the synergistic combinations, the chimeric and conjugated molecules and the improved antibiotic derivatives of the present invention for preparation of medicaments for prevention and treatment of bacterial infection is also within the scope of the present invention as well as methods for producing such combinations, chimeric, conjugated and improved molecules,.

The synergistic combinations, chimeric, conjugated and improved molecules of the present invention are used, according to yet another aspect, for prevention and treatment of bacterial infection.

Methods for prevention and treatment of bacterial infection using the synergistic combinations, chimeric, conjugated and improved molecules of the present invention represent another aspect of the present invention.

According to some embodiments, the antibiotics of the present invention are active against gram-positive bacteria.

The compounds of the present invention may be produced using biological, bio-synthetic, synthetic or semi-synthetic methods known in the art. Typically, antibiotics and antibiotic derivatives are produced by microorganisms and isolated using methods known in the art. Peptide-based compounds may be synthesized using any method know in the art, including solid-phase peptide synthesis methods.

According to some aspects of the present invention, crystallographic analysis provides insight into the minute structural differences between similar and different antibiotics that can turn competition into synergism, hence providing criteria for development of synergistic antibiotics having improved clinical potential.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1: Chemical structures of Streptogramin antibiotics. Three pairs of molecules are shown, in each the compound that bind to the PTC are in the upper panel, and their mates that bind to the NPET are in the lower panel immediately below them. Erythromycin is inserted for size and structure comparisons.

FIG. 2: Modeling of LC (A), LM (B) and LC and LM bound together (C) in their unbiased (Fo-Fc) difference electron density map (observed structure factors minus the calculated structure factors as coefficients for electron density maps), contoured at 1.0σ around the antibiotics. In grey ribbons the surrounding rRNA backbone is shown.

FIG. 3: Chemical Interactions of lankacidin and lankamycin with their D50S binding pockets (dashed bonds indicated hydrogen bond). (A) Interactions of lankacidin with the surrounding rRNA (thin lines). (B) Interactions of lankamycin with the surrounding rRNA (thin lines). (C) Interactions of lankacidin and lankamycin with the surrounding rRNA (thin lines).

FIG. 4: Structural overlay of binding site around Ery (in black) and LM (in grey) (ery coordinates are taken from Ery-H50S complex (1YI2) with Lankamycin-D50S complex, note the shift of A2062 upon LM binding compared to its position in an empty subunit and to its position when Ery and LM/LC bind.

FIG. 5: Effect of LC on (A) protein synthesis in the E. coli cell-free system and (B) peptide bond formation catalyzed by S. aureus 70S ribosomes (circles) or D. radiodurans large ribosomal subunits (squares). C. Competition of LC with ¹⁴C-ERY for binding to D. radiodurans 50S subunits. (D) Inhibition of cell-free translation (E. coli) by LM

FIG. 6: Chemical probing (“footprinting”) of interactions of LC (filled arrowheads), LM (open arrowheads) and ERY (open circles) with the ribosome. Protection of 23S rRNA residues from (A) CMCT and (B) DMS modification by LC. C. Protection of 23S rRNA residues from CMCT modification by LM. E. Protections from CMCT afforded by LC, LM, and ERY present alone or in combination. Small open circles indicate bands that appeared because of slight nuclease degradation, which were not reproducible between the repeated experiments.

FIG. 7: Synergistic inhibitory activity of LC and LM upon S. aureus. The plots represent changes in minimal inhibitory concentrations (MIC) of individual compounds when both drugs are present in combination. The general shapes of the hypothetical curves corresponding to the additive, synergistic or antagonistic mode of the drug combinations are shown by broken lines. The experimental curve is shown as a solid line with the MIC values (shown as a fraction of MIC of LM or LC acting alone) indicated by filled circles.

FIG. 8: A and B: Binding curves for LC and LM respectively. The production of green fluorescent protein in an in vitro transcription-translation reaction was used to determine inhibition. C: Synergy experiment showing the fractional contributions towards the observed C50 by LC and LM. Points below the dashed line indicate synergism.

### DETAILED DESCRIPTION OF THE INVENTION

The present invention is based in part on the novel findings disclosed herein for the first time that two antibiotics, LC and LM, produced by S. rochei are protein synthesis inhibitors that act synergistically to produce a synergistic anti-bacterial effect.

The sites of binding and the modes of action of LC and LM were identified by crystallographic and biochemical analyses. It is shown that these two compounds bind at neighboring sites in the large ribosomal subunit, which partially overlap with the binding sites of two components of streptogramin antibiotics. It is further shown that LC can bind simultaneously with LM and that the two drugs inhibit bacterial growth synergistically, suggesting that the structures of LC and LM allow simultaneous cooperative action. Based on the structural results presented herein, means for enhancing the synergetic inhibitory effect of these compounds, as well as of other antibiotic molecules are provided, as well as improved compounds and combinations designed using the novel structural results.

The structural and biochemical data disclosed herein support the concept that optimization of this drug pair leading to high affinity concurrent binding to the ribosome may be reached by specific chemical alterations or conjugation. Furthermore, the results suggest that certain combinations of peptidyl transferase center (PTC) inhibitors with the nascent peptide exit tunnel (NPET)-bound compounds in their natural or chemically modified derivatives exhibit synergy. The present invention thus provides potent antibiotic drugs and drug combinations by co-optimizing their structure or by linking them together into a single molecule for the development of advanced antibiotics targeting the ribosome.

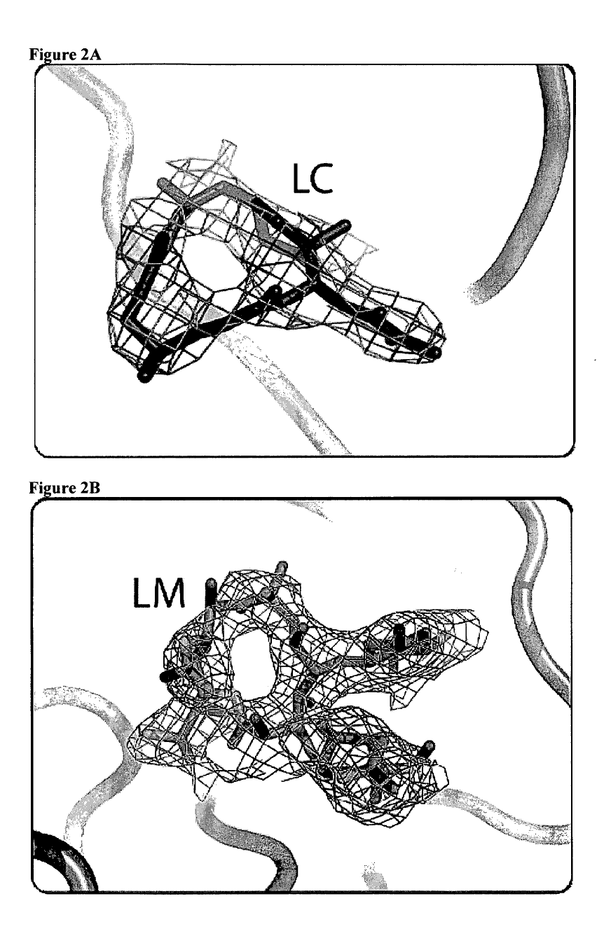

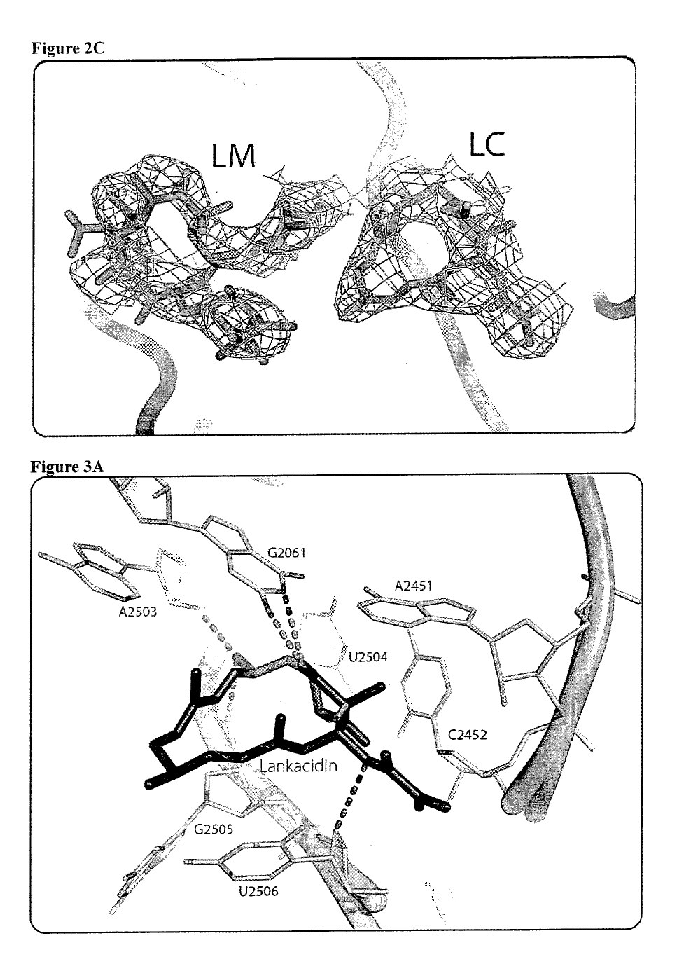

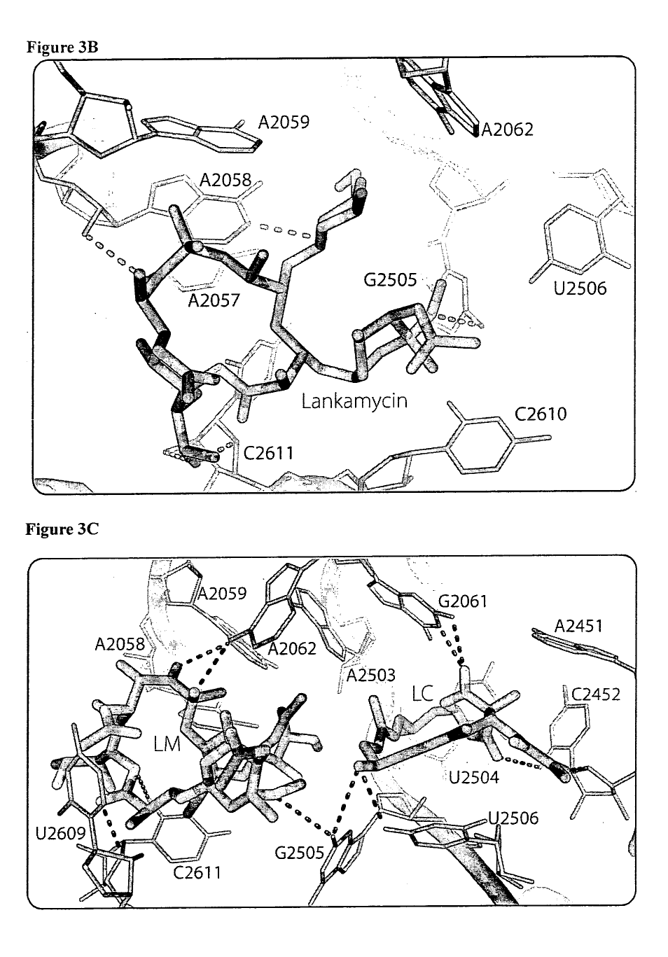

Complete sets of X-ray diffraction data were collected for the D50S complexes with lankacidin (LC-D50S), the D50S complexes with lankamycin (LM-D50S) and with both lankamycin and lankacidin (LM/LC-D50S) to a maximum resolution of 3.5, 3.2 and 3.45 Å respectively. Using isomorphous replacement to obtain initial phases, clear electron density was observed for the binding sites of LM in LM-D50S and LM and LC in LM/LC-D50S, allowing unambiguous determination of the binding sites of the antibiotics (FIGS. 2 and 3).The crystallographic and biochemical data presented here firmly established the PTC as the site of LC action. The inhibitory effect of the antibiotic upon peptide bond formation is likely achieved by preventing the binding or the proper placement of aminoacyl moiety of aminoacyl-tRNA in the A site. As LC trespasses the P site, it may also affect the exact positioning of the peptidyl tRNA C-terminus. Although LC is chemically distinct and is less bulky than most S_{A}-type drugs, its binding site partially overlaps with that of S_{A} compounds (Hansen, J. L. et al. ibid, Harms, J. et al. ibid). Furthermore, both, LC and S_{A} drugs shift the orientations of U2585 and U2506 that are involved in PTC functions.

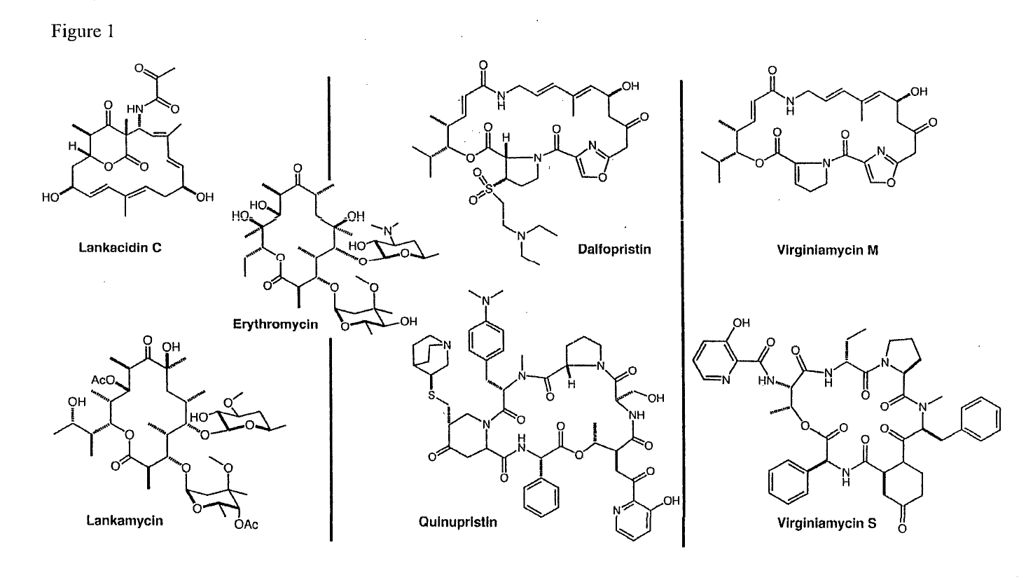

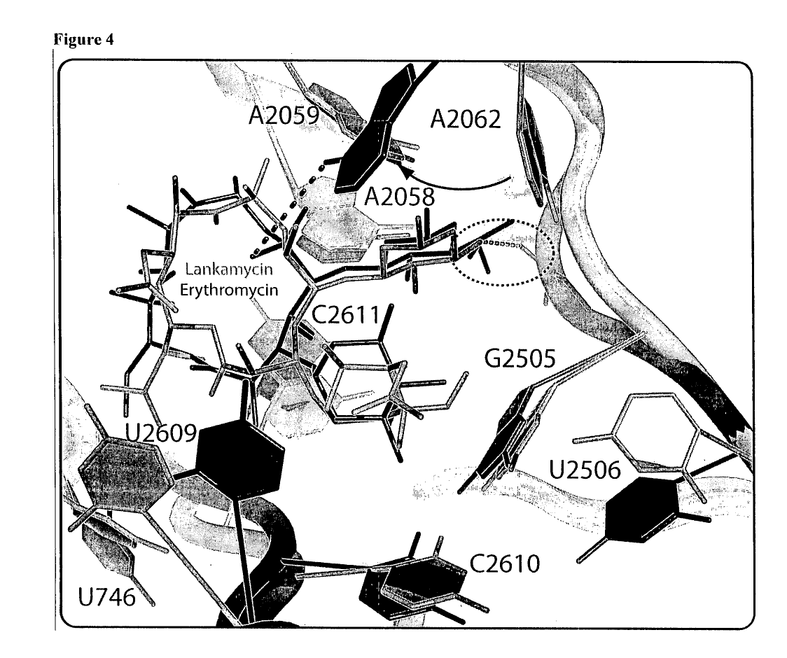

X-ray crystallographic analysis of the ribosomal complexed with LM alone and in conjunction with LC further revealed that the binding mode and position of LM was extremely closely related to the archetypical macrolide, erythromycin. LM binds to the NPET macrolide binding site in a fashion similar, albeit not identical, to the other macrolides (FIG. 4). The same NPET region accommodates the B components of streptogramin antibiotics, but owing to differences in chemical nature (FIG. 1), the S_{B} compounds and macrolides exploit a different set of interactions (Tu, D. et al., ibid).

Since the LC and S_{A} binding sites in the PTC (FIG. 3C) are adjacent to the macrolides and S_{B} binding sites, it is conceivable that compounds acting upon these two sites can either compete or cooperate in binding to the ribosome. Streptogramins are known for their synergistic action (Cocito, C. et al., ibid, Porse, B. T. and Garrett, R. A., 1999, J Mol Biol 286, 375-387). Such cooperativity makes evolutionary sense: the same microorganism produces both S_{A} and S_{B} components and their mutually enhanced action should be highly beneficial for the antibiotic producer. This notion initially did not seem to hold true for LC and a macrolide LM. The experiments showed that LC and ERY do not cooperate, but rather compete for binding to the D. radiodurans ribosome. Hindrance might result from a direct clash between ERY desosamine sugar and the LC macrocyclic ring or by allosteric modulation of the binding site of one compound by binding of its counterpart. The altered DMS reactivity of A2058 and A2059, the nucleotides important for macrolide binding (FIG. 3B and 3C), upon the LC binding, might be a reflection of such allostery. LC and ERY are produced by different microorganisms and were not ‘designed’ to work together. In contrast, LM is co-produced with LC and both drugs appear to be co-regulated, similar to the streptogramins case, seem to be intended to work together (Arakawa, K. et al., ibid). In agreement with this hypothesis, the results show their simultaneous binding to the ribosome. The small structural differences between ERY and LM (FIG. 1) are likely to cause the variation in their binding properties. One of the important distinctions between LM and ERY is the nature of the C5-linked sugar residue (D-desosamine in ERY vs. D-chlacose in LM). Even small modifications of desosamine in ERY can dramatically alter the antibiotic's activity (Wu, Y.J. and Su, W.G., 2001, Curr Med Chem 8, 1727-1758; Yonath, A., 2005, Annu Rev Biochem 74, 649-679). Replacement of 3′ dimethyl amine of ERY with a methoxy group in LM may facilitate accommodation of the C5-sugar residue in a narrow space left between the A2058/A2059 ridge and the bound LC molecule (see FIG. 3C). Similarly, minute modifications in the structure of the antibiotics or in their binding sites may have significant effects on their binding and properties (Berisio, R. et al. ibid, Vazquez-Laslop, N. et al., 2008, Mol Cell 30, 190-202). LM on its own is a less potent protein synthesis inhibitor than ERY. However, LM reduced activity is compensated by its ability to act synergistically with LC. Thus, it appears that the LM synthetic pathway has been evolutionary optimized to generate a 14-member ring macrolide capable of simultaneous binding and synergistic action with LC. It is hardly a coincidence that two different combinations of synergistic protein synthesis inhibitors, the S_{A} and S_{B} components of streptogramins or LC and LM, utilize the same two adjacent sites in the large ribosomal subunit. These sites might be best suited within the ribosome for accommodating pairs of compounds that would be able to tightly bind and inhibit protein synthesis in a synergistic fashion. Previously, significant resources went into development of streptogramins into a clinical drug. This effort resulted in a useful and successful streptogramin antibiotic, Synercid. Yet, no focused attempt was dedicated to optimizing the combination of LC/LM, or for that matter, LC with any other macrolide. The present studies demonstrate the validity of LC/LM synergism and provide a structural basis for chemical modifications of either of the two components, which could lead to the improvement of the inhibitory action of the LC/LM pair and its clinical relevance. Notably, the S_{A} compounds (for example quinupristin or virginiamycin S) are significantly larger than LC (FIG. 1) and therefore have more chemical entities facilitating their binding to the ribosome pocket and forming stable network of interactions. It is likely, therefore that decorating LC with additional groups capable of forming new interactions with rRNA in its binding site may increase its affinity. Modulating the properties of the LM sugar residues may further add to the potency of the LC/LM pair.

### Lankacidin Binding Site

The 3.5 Å resolution (Table 1) difference electron density map calculated between the structure amplitudes of the large ribosomal subunits from Deinococcus radiodurans (D50S) in complex with LC (D50S-LC) and of the D50S native structure (41) allowed the unambiguous determination of the location and conformation of LC in the PTC (FIG. 2A).

LC binding pocket is composed of nucleotides A2602, C2452, A2503, U2504, G2505, U2585, G2061 and U2506 (E. coli numbering throughout) and the bound LC is involved in an extensive network of hydrophobic interactions with most of these nucleotides. Additionally, LC is positioned within hydrogen bond distance to 2′-OH and the exocyclic amino group of G2061, the ribose hydroxyls of A2503, of G2061 and 05′ of G2505 (FIG. 3A). It partially occupies the location of the amino acid attached to the 3′ end of A site tRNA (FIG. 3A) and barely reaches the macrolide binding site. The re-positioning of the rRNA residues that occurs as a consequence of LC binding creates a unique network of interactions between five re-oriented nucleotides: U2506, G2505, G2581, C2610 and G2576. Within this network G2576 stacks upon G2505, C2610 stacks upon G2581, and the exocyclic amine of G2505 is at a hydrogen bonding distance from O2 of C2610. Also, the amino group of G2502 is within a hydrogen bond reach of O4 of U2506, which shifts towards the LC's 1,3 dicarbonyl system. These newly established contacts stabilize the placement of G2505 and U2506 in a conformation that favors binding of LC. Additionally, similar to pleuromutilins that utilize a network of remote interactions, LC binding is influenced by the second-shell nucleotides, specifically G2576, A2062, C2530, U2531, C2507, U2584, G2581, C2610 and A2059. Thus, LC exploits the PTC inherent flexibility for achieving high binding affinity (Davidovich, C. et al. ibid).

In its binding site, LC macrolactone ring fits in the shallow depression in the wall of the PTC A-site, by forming van der Waals interactions with U2504, G2505 and U2506. The importance of these interactions to the drug binding is manifested by structure-activity studies that showed that hydrogenation of the macrocyclic ring alters its ring conformation, and reduces the inhibitory activity of LC (Hansen, J. L. et al., 2002, Mol Cell 10, 117-128). The 2-methyl group at the lactone edge of the macrocyclic ring inserts in the opening of the hydrophobic crevice formed by the splayed out bases of A2451 and C2452. This cleft also hosts the aminoacyl moiety of A site-bound aminoacyl-tRNA, and is involved in binding of other PTC-targeting antibiotics, including S_{A} antibiotic compounds such as dalfopristin and virginiamycin M (Gurel, G. et al. ibid, Bashan, A. et al., 2003, Mol Cell 11, 91-102). The overlap of the LC and chloramphenicol binding sites provides the structural basis for their competition for binding to the ribosome.

Despite significant size differences (FIG. 1), the position of LC closely resembles those of dalfopristin and virginiamycin M. However, substantial differences were observed in the interactions of these compounds with the ribosome. In the D50S-Synercid complex dalfopristin ring extends significantly farther towards the P-site. As LC binds in the PTC center, it causes the flexible base of A2602, which plays a major role in tRNA translocation, to undergo a 45° rotation compared to its placement in native D50S or in Synercid-bound complex. U2585, the second flexible nucleotide that also seems to play a role in A-tRNA translocation, undergoes only a minor alteration in D50S-LC complex, while it is rotated by 180° in D50S-Synercid complex. This rotation seems to occur because of steric hindrance of the dalfopristin large macrocyclic ring and to the occupation of the S_{B} site by quinupristin, the S_{B} component of Synercid. Other important details distinguish binding of LC and dalfopristin. While both drugs are hydrogen-bonded to the G2061 exocyclic amine, an additional H-bond links dalfopristin with G2061 2′ hydroxyl. Both LC and Synercid induce a conformational change of C2610. However, in the D50S-LC complex C2610 stacks upon G2581 and is H-bonded to G2505, whereas in the D50S-Synercid complex it is flipped away because of steric hindrance caused by quinupristin. Additionally, while both LC and dalfopristin re-orient the U2506 base, the shift of this base toward LC is unique.

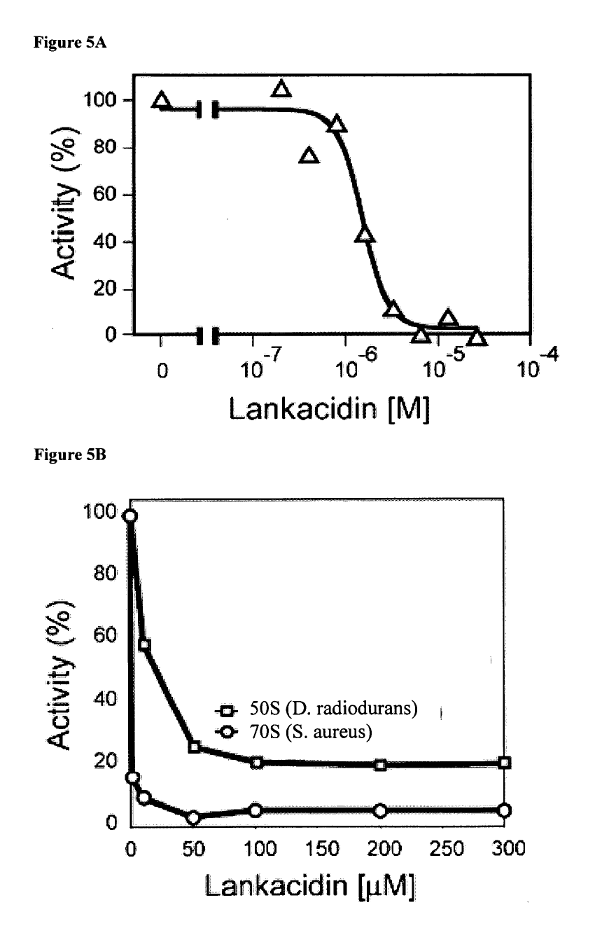

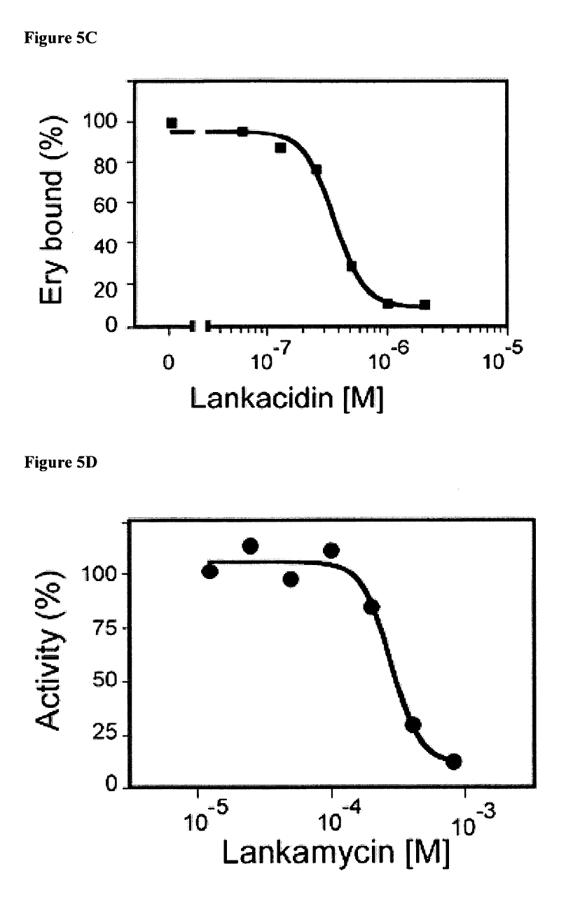

Consistent with its binding to the functionally critical PTC, LC inhibited bacterial (E. coli) cell-free transcription-translation system, with a respectable IC₅₀ of 1.5±0.1 μM (FIG. 5A). Furthermore, it was found that the drug readily interfered with the peptide bond formation inhibiting the puromycin reaction catalyzed by either Staphylococcus aureus 70S ribosomes (IC₅₀ 0.32±0.02 μM) or isolated large ribosomal subunits of D. radiodurans (IC₅₀ 10.0±6.0 μM) (FIG. 5B). This result affirmed LC as an effective PTC inhibitor.

TABLE 1
Crystallographic data for the D50S-lankacidin complex

| Parameters | |
|---|---|
| Space group | I222 |
| Resolution (Å) | 40-3.5 (3.63-3.5) |
| R_{sym}(%) | 16.3 (82.7) |
| Completeness (%) | 92.4 (91.8) |
| Redundancy | 5.3 (4.6) |
| I/σ(I) | 7.7 (1.5) |
| Unit Cell (Å) | a = 169.8 b = 410.3 c = 694.4 |
| R/R_{free}(%) | 26.8/32.4 |
| Bond length (Å) | 0.006 |
| Bond angles (degrees) | 1.185 |

Table 2 summarizes the [IC50] values of several antibiotics, for antibiotic inhibition of cell free translation.

TABLE 2
Comparison of [IC50] values for antibiotic
inhibition of cell free translation.

| Antibiotic | [IC50] (μM) |
|---|---|
| Lankamycin (LM) | 275 |
| Lankacidin (LC) | 1.5 |
| Erythromycin (Ery) | 0.2 |
| Virginiamycin M | 0.8 |
| Virginiamycin S | 2.5 |
| Synercid ™ | ~0.1 |

### Lankamycin Binding Site

The binding site of LM in the 50S subunit is located at the nascent protein exit tunnel near its entrance. In its binding site LM makes contacts with rRNA nucleotides and not with any r-proteins. It forms hydrogen bond contacts between the chalcose sugar and A2058 (E. coli numbering used throughout text), the ketone on C-9 and 2′-OH on A2058 and a bridging polar contact between the hydroxyl pendant of C-13 and the ribose sugar of C2611 (FIG. 3B). The other contacts it makes with the ribosome are hydrophobic interactions with A2059, A2062, G2505, U2506, C2510 and C2611, completing the binding pocket for LM.

Upon binding of LM there is a significant change in position of the flexible nucleotide A2062 (important in other synergetic antibiotic interactions, see below) away (compared to the unbound conformation) from the antibiotic. Presumably this is due to the steric hindrance of the methyl group C-6 of the macrolide ring, which would be in close contact with the exocyclic amine of A2062. This base movement causes a slight change in the conformation of the nucleobases surrounding A2062. Interestingly it is A2062 that forms a significant hydrogen bond with a related macrolide erythromycin (Ery) between the hydroxyl group at C-6 on Ery and the exocyclic amine on A2062 (FIG. 4). Notably, in the Ery-H50S structure the conformation of A2062 is similar to the native position of the unbound ribosome (FIG. 4). Upon macrolide binding there is a significant rearrangement of the position of C2610, a nucleotide that is located at the opening of the exit tunnel. In its native conformation, C2610 would sterically clash with the macrolides (LM and Ery, FIG. 4), however in the presence of the antibiotics it moves away from the tunnel.

The [IC₅₀] of LM (Table 2), shows that compared to Ery it exerts a weaker inhibition of ribosomes by a few orders of magnitude. A comparison of the structures of LM-D50S complex with the Ery-50S complex, shows that LM, while binding in the same binding pocket does not benefit from two of Ery's main interactions, a hydrogen bond to A2062, and a salt bridge that Ery makes between its protonated amine on the desoamine sugar and the phosphate oxygen of G2505. This coupled with the significant rRNA rearrangements (due to A2062 rotation) upon LM binding in the exit tunnel, yields insight as to the reasons for the weaker binding of LM to the 50S subunit compared to Ery.

LM/LC-D50S Binding Site

The structure of both the LM and LC in complex with the 50S subunit reveals that they bind in same general sites that each uses when they are separately bound. LM binds in a similar manner, although it translates slightly closer to the PTC (˜1 Å), whereas LC resides in an almost identical position (FIGS. 2C and 3C). Both antibiotics make similar contacts with the ribosome, contacting only rRNA or each other.

LM makes hydrogen bond contacts between the ribose C2611 but due to its slight translation it now forms a hydrogen bond with endocyclic nitrogen in A2059 instead of A2058. The arcanose moiety also forms a new hydrogen bond between the acetate oxygen and U2586. A2062 is in a similar binding position as in the native D50S and Ery complex structures, forming a new hydrogen bond between the exocyclic amine and the hydroxyl group on C-8 of the macrolactone ring. Interestingly, G2505 forms a bridging hydrogen bond between the two antibiotics (see FIG. 3C). There are also very similar hydrophobic interactions with surrounding bases within the binding pocket of LM being completed by A2058, A2503, U2609 and C2610.

LC resides in almost exactly the same binding site as in its native LC-D50S complex. It makes hydrophobic contacts with A2451, C2452, U2504, U2585 and hydrogen bonds between A2053, G2061, C2452 and U2506 (FIGS. 3B and 3C). There is also the bridging polar contact to LM mediated by G2505. LC binds in a well-defined Mg^{II} binding site, with the ketoamide group displacing the native position of the Mg^{II}, suggesting that the binding of this antibiotics is actually responsible in some manner for mediating the rRNA stability in this region.

Binding of LC into the PTC also induces some coordinated changes in the position of several rRNA nucleotides. There is coordinated movement of U2585 and U2506 (FIG. 3C), wherein upon binding of LC, U2585 moves away from LC to avoid a steric collision. This movement induces a change in the position of U2506 as it is no longer sterically hindered by U2585, this movement is also confirmed by 23S rRNA footprinting studies (vide infra).

LC and LM also make multiple van der Waals contacts between them. The arcanose and chalcose rings of LM are within hydrophobic contact with the conjugated section of the LC macrolactone ring. These interactions would not be achieved unless LM also moved closer to the PTC as it does in the double antibiotic structure.

Crystallographic refinement details of LM-D50S and LM/LC-D50S are presented in Table 3.

TABLE 3
Crystallographic collection and refinement parameters.

| Parameters | LM-D50S | LM/LC-D50S |
|---|---|---|
| Crystals Merged | 4 | 8 |
| Osc Angle (φ°) | 0.3° | 0.2° |
| Beam Line | ESRF 23-2 | SLS-PXI |
| Detector | MARCCD-225 | Pilatus-6M |
| Resolution (Å) | 35-3.20 (3.31-3.20) | 35-3.45 (3.57-3.45) |
| R_{merge} (%) | 17.8 (71.3) | 21.6 (85.7) |
| Completeness (%) | 93.2 (34.0) | 84.3 (85.6) |
| Redundancy | 3.7 (2.2) | 4.6 (4.5) |
| I/σ | 7.1 (2.4) | 5.6 (1.4) |
| Space Group | I222 | I222 |
| Unit Cell (Å) | a; 170.6 | a; 169.7 |
| | b; 410.2 | b; 408.6 |
| | c; 695.1 | c; 693.3 |
| R_{work}/R_{free} (%) | 25.4/29.4 | 23.5/29.1 |
| rmsd bonds, (Å) | 0.01 | 0.01 |
| rmsd angles, (°) | 1.1 | 1.3 |

### Lankacidin and Lankamycin Can Simultaneously Bind to the Ribosome

S. rochei secretes two antibiotics, a 17-member ring macrocyclic LC and a 14-member ring macrolide, lankamycin (LM). LM is structurally similar to ERY (FIG. 1) and thus it was assumed that it is likely to bind to the ribosome at the site and orientation similar to ERY, namely at the NPET in immediate proximity to the PTC, the LC binding site. However, the comparison of the position of ERY in D50S with the crystal structure of D50S-LC complex (FIG. 4) revealed that the desosamine sugar of ERY approaches the macrocyclic ring of LC too close for simultaneous binding of both drugs. In agreement with this notion, competition experiments showed that LC displaced ¹⁴C-ERY from D50S with IC₅₀ of 355±26 nM (FIG. 5C). These observations raised the question whether, similarly to ERY, LM would also compete with LC for ribosome binding.

To address this question, it was first verified that LM binds to the ribosome and inhibits protein synthesis. Indeed, in the E. coli cell-free system, LM inhibited translation (IC₅₀ of 275±36 μM) (FIG. 5D) arguing that the antibiotic does bind to the ribosome, albeit with only moderate affinity. RNA probing was then utilized to follow the binding of LC and LM to the ribosome. For the consistency of structural data, RNA probing experiments were carried out using D. radiodurans large ribosomal subunits. Binding of LC and LM was analyzed using chemical modifying reagents 1-cyclohexyl-3-(2-morpholinoethyl) carbodiimide metho-p-toluene sulfonate (CMCT) and dimethyl sulfate (DMS).

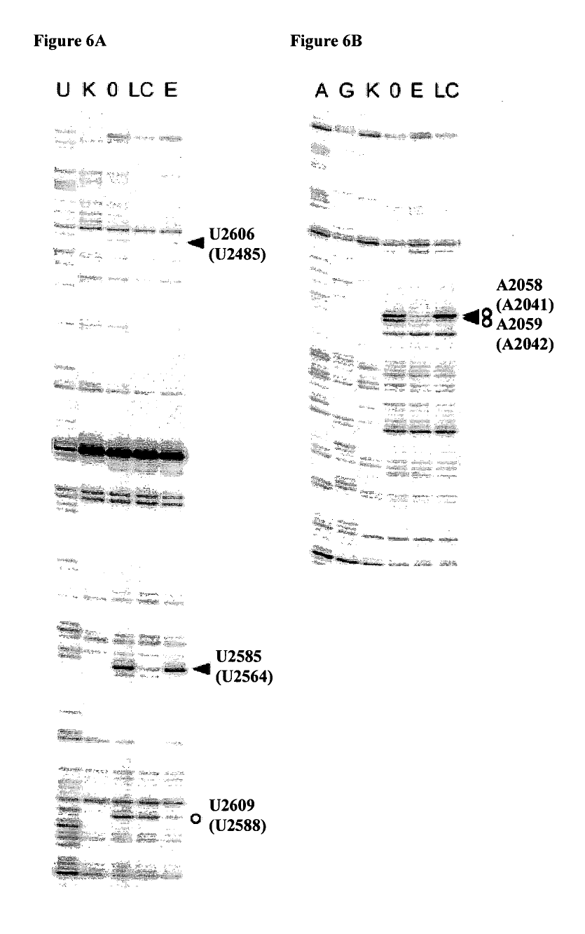

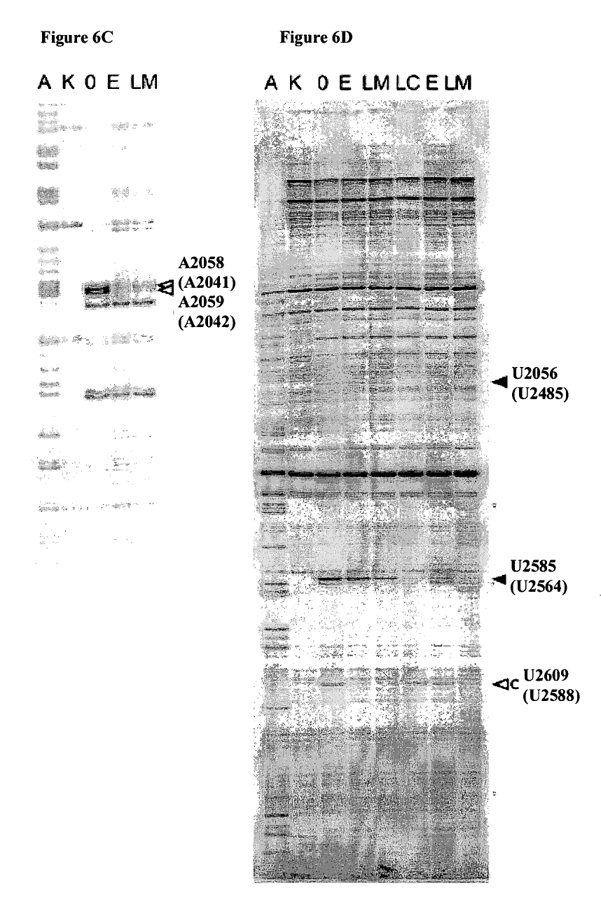

In accord with crystallographic data, association of LC with D50S results in a strong protection of the PTC nucleotide residues U2506 and U2585 from CMCT modification (FIG. 6A). It is also noted that upon LC binding, A2059 became partially protected from DMS whereas modification of A2058 was increased (FIG. 6B). This effect correlated with the crystallographic structure of the LC-D50S complex where upon LC binding A2059 is stacked upon A2503 and such stacking may partly expose the surface of A2058, yielding access for DMS. This result indicated that LC-induced re-structuring of the D50S nucleotide residues in the PTC can propagate allosterically to the proximal segment of the NPET.

23S rRNA High Resolution Chemical Footprinting Studies

Similar to ERY and other 14 member-ring macrolides, binding of LM results in protection of U2609 from CMCT modification and of A2058 and A2059 from DMS chemical modification performed on the 23S rRNA of D. Radiodurans in complex with the combinations of the antibiotics (FIGS. 6C,D). The idiosyncratic protections of 23S rRNA residues were exploited against CMCT modification, afforded by LC and LM, to interrogate their simultaneous interaction with the ribosome. At 50 μM, LC shields U2506 and U2585 from alterations. However, the accessibility of these residues to CMCT is not affected by LM or ERY (FIG. 6). Conversely, LM (at 500 μM) or ERY (at 50 μM) strongly protect U2609, while LC has no effect on this nucleotide. Thus, protection of U2506 and U2585 indicates LC binding, whereas protection of U2609 reveals LM or ERY binding. When LC and LM are present together, all the three residues (U2506, U2585 and U2609) are protected, indicating that LC and LM are simultaneously bound to the ribosome. In contrast, and in agreement with the binding experiments, the LC-mediated protection of U2506 and U2585 is partially relieved upon addition of ERY (FIG. 6D) and ERY-dependent protection of U2609 is partly reversed when LC is present. Thus, while LC and ERY compete for the binding to the ribosome, LC and LM can bind simultaneously to their respective targets in the PTC and NPET.

When added alone, LM protects A2059 and A2058 from DMS modification and U2609 from CMCT modification in the same manner that Ery does. The crystallographic data of the LM and LC/LM combination, confirms this observation, showing overlap between Ery and LM positions (FIG. 4). LC partially protects A2059 as in the case of LM, yet it causes a hypersensitive response to DMS modification on A2058. Curiously, neither A2058 nor A2059 are in the binding vicinity of LC, but drug binding in the region of the PTC and early exit tunnel region desensitizes these bases from chemical cleavage. This may suggest that these bases are involved in induced fit mechanism, conferring protection. However, there is no structural evidence for large rearrangement of these bases compared to their native conformation. More likely, any drugs binding in this region (PTC, exit tunnel) act as simple steric inhibitors for DMS or CMCT, disallowing chemical cleavage.

When in complex (LC/LM), an intermediate effect is observed, as modification of A2058 is weaker, in agreement with the simultaneous binding of the drugs. This intermediate effect is visible with the flexible U2585. LM and Ery do not affect the accessibility of U2585, and LC alone shields U2585 from alterations. However, in complex with LM this shielding is partially relieved.

U2585 is part of the LC binding pocket, situated in proximity of the LC ketoamide group. The Mg^{II} ion in the native D50S structure (also in D50S-LM) also creates a hydrogen bond with U2585, and is displaced by LC. This observation can also explain the solvent accessibility of U2585 as the Mg^{II} does not hinder modification by CMCT.

### LC and LM Act Synergistically Upon Bacterial Cells

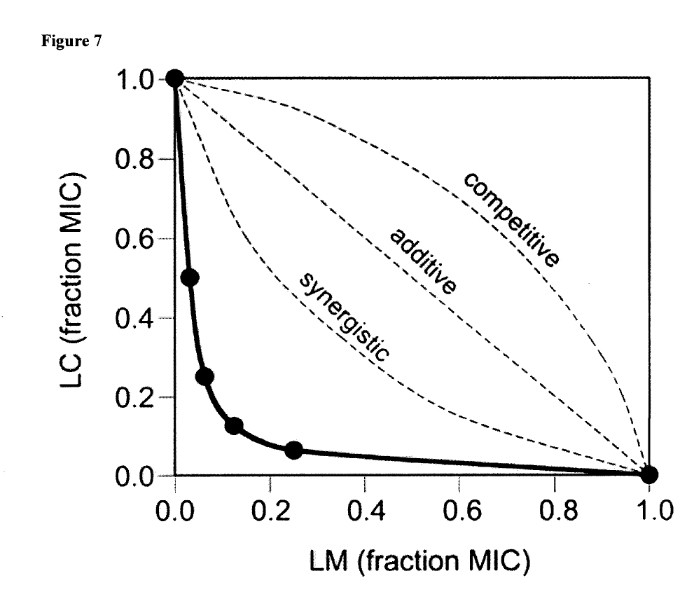

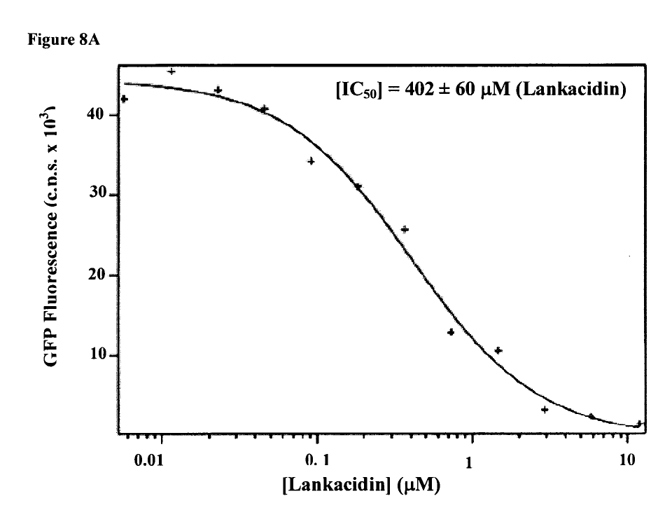

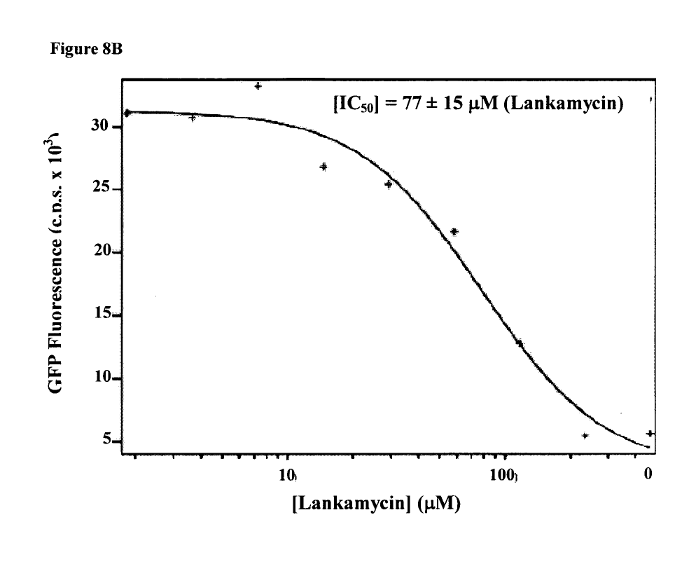

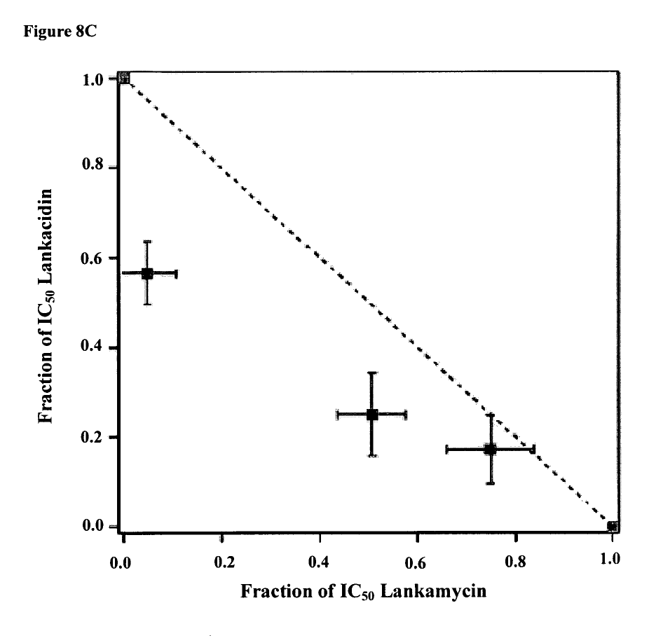

As the binding site of LC and LM partially overlaps with that of S_{A} and S_{B} antibiotics, and as S_{A} and S_{B} act synergistically, it was hypothesized that despite the difference in their size and chemical properties from streptogramins, LC and LM may also exhibit synergy. To address this issue, in vivo and in vitro experiments were performed, using whole cell bacteria as well bacterial cell-free system. Synergism was observed by an in vivo assay that utilizes a susceptible strain of Gram-positive S. aureus. The minimal inhibitory concentration (MIC) was analyzed in a checkerboard fashion and the results were plotted as fraction of MIC (FIC) of individual compounds (FIG. 7). In this assay, antibiotics are considered synergistic if the curve has a concave shape, whereas a linear plot reflects additive action of the drugs and a convex graph shows antagonistic interaction. The experimental MIC plot (solid line in FIG. 7) had a well-pronounced concave character revealing synergy in action of LC and LM. These findings were further verified in E. coli cell-free transcription-translation system. Thus, similar to streptogramins, the two antibacterial compounds produced by S. rochei bind simultaneously to the neighboring sites in the ribosome and synergistically inhibit sensitive bacteria.

The crystallographic structure determination the 505 ribosomal subunit in complex with LC, LM and LM with LC gives a clear indication as to their mechanism of ribosomal inhibition. LM binds in a very similar manner to other members of the macrolide antibiotic family, essentially overlapping with the binding site of erythromycin in the exit tunnel. Like other members of the family, LM action arises from physically blocking progression of the nascent peptides through the tunnel. The binding site of LC in complex with LM indicates drug binding in the PTC, preventing the proper placement of the aminoacyl end of the A-Site tRNA.

A comparison of the structural overlay of the other synergetic pair of Streptogramins clearly shows that both components bind to a similar region of the ribosome. LM and Quinupristin (Qn) bind in the exit tunnel and LC and Dalfopristin (Dn) are located in the PTC. In fact there are many similarities in their modes of action; both make hydrophobic contacts with each other, both make bridging contacts with the same nucleotide (A2602 for Qn/Dn and G2505 for LM/LC) and both bind the 50S subunit making contacts solely with rRNA and their binding is facilitated by an induced fit mechanism.

### Materials and Methods

Crystals of D50S, grown as in (Harms, J. et al., 2001, Cell 107, 679-688), were soaked in a solution containing lankacidin. Crystallographic data were collected with highly collimated synchrotron X-ray beam and processed with HKL2000 and (CCP4, 1994, Acta Crystallogr D Biol Crystallogr 50 (Pt 5), 760-763) using the available crystal structure of lankacidin C (Uramoto, M. et al., 1969, Tetrahedron Lett 27, 2249-2254). After map tracing and refinement by COOT and CNS ribosome-antibiotic interactions were identified by LigPlot and LPC and images were generated by PyMol. Coordinates were deposited in the protein data bank (PDB), with accession code 3JQ4.

Antibiotic binding and inhibition of cell-free translation were determined as described in (Xiong, L. et al., 2005, Antimicrob Agents Chemother 49, 281-288). Inhibition of the peptidyl transferase reaction by LC was performed and analyzed as described in. The data were analyzed using Prism 4 (GraphPad Software). RNA probing was carried out as described in (Moazed, D. and Noller, H. F., 1987, Biochemie 69, 879-884) using D50S.

### Crystallography

For LC: Crystals of D50S that were grown as in (Harms, J. et al., 2001, ibid) were soaked in solutions containing 0.025 mM of lankacidin for 20 hours at 20° C., transferred into cryo-buffer and shock-frozen in liquid nitrogen. X-ray data were collected at 85-100 K from shock-frozen crystals at wavelength of ˜1.0 and 0.837 Å, at crystal to detector distance of 430 mm, using an oscillation range of 0.3° with synchrotron radiation beam at 191D at the Advanced Photon Source/Argonne National Laboratory and at ID23-2 at the European Synchrotron Radiation Facility (ESRF), respectively. Data were recorded on charge-coupled device and processed with HKL2000. Complete X-ray data sets were obtained from two crystals. The structure of D50S was refined against the structure factor amplitudes of the antibiotic complex D50S-LC using rigid body refinement as implemented in CNS. For free R-factor calculation, random 5% of the data were omitted during refinement. To obtain an unbiased electron density map a composite omit map of the entire unit cell was calculated. Further refinement was carried out using CNS 1.2 minimization combined with various programs, exploiting the available crystal structure of Bundlin-A (lankacidin, Uramoto, M. el al. ibid).

Finally, the complete molecules were subjected to restraint minimization and grouped B factor refinement with CNS. The ribosome-antibiotic interactions were determined with LigPlot and LPC. Images were generated using PyMol.

For LM and LC/LM complex: D50S were isolated and crystallized as previously described (41). The crystals of the LM-D50S complex were soaked in a solution of HEPES (pH=7.8, 21° C., 10 mM), MgCl₂ (15 mM), ammonium chloride (75 mM), ethanol (20% v/v), 2-ethyl-1,3-hexanediol (10% v/v) and lankamycin (900 μM), for 6 hours prior to flash freezing. Crystals of the LM/LC-D50S complex were grown in the presence of lankamycin (400 μM) and were subsequently soaked in the same buffer conditions as above with the addition of lankacidin (25 μM).

Diffraction data were collected using a highly collimated synchrotron X-ray beam, using thin slice phi oscillation scans. Data were processed using MOSFLM, HKL2000 and CCP4. Map tracing, phase and model refinements were performed using COOT, CNS and PHENIX. Densities for the antibiotics were located on both a standard Fourier difference maps as well as simulated annealed composite-omit maps. Chemical restraints for the antibiotics were prepared using the PRODRG server (http://davapc1.bioch.dundee.ac.uk/prodrug/) and were fitted to the difference maps. Mg²⁺, Na⁺ and K⁺ ions were located manually by careful analysis of the Fourier difference map, no attempt was made to model discrete water molecules. The antibiotic interactions with the ribosome were examined using LigPlot and all images were generated by PyMOL.

### Antibiotic Binding

D. radiodurans 50S subunits (0.1 μM) were preincubated 15 min at 37° C. with 0.1 μM of ¹⁴C-erthromycin (48.8 mCi/mmol, Perkin Elmer) in 100 μl binding buffer (20 mM Tris-HCl, pH 7.5, 10 mM MgCl₂, 250 mM NH₄Cl, 6 mM β-mercaptoethanol). LC was then added at varying concentrations (0-2 μM) and incubation continued for 30 min. The ribosome-antibiotic complexes were purified by gel filtration in BioGel P30 spin columns as described (Xiong, L. et al. ibid) and the amount of ribosome-bound radioactivity was measured by scintillation counting.

### Inhibition of Cell-Free Protein Synthesis by LC and LM

The E. coli cell-free transcription-translation system for circular DNA (Promega) was pre-incubated with varying concentrations of antibiotics for 5 min at 20° C. The reactions (10 μl final volume) were initiated by adding 0.64 μg of pBestLuc plasmid DNA (Promega). Reactions were incubated for 40 min at 20° C. and stopped by chilling on ice. The activity of firefly luciferase synthesized in the reaction was determined in 96-well plates using Bright-Glo Luciferase Assay System (Promega) as recommended by the manufacturer.

### Inhibition of the Peptidyl Transferase Reaction by LC

In the reaction catalyzed by large ribosomal subunits, D. radiodurans 50S subunits (200 nM final concentration) were combined in 50 μl of the reaction buffer (20 mM Tris-HCl, pH 8.0, 20 mM MgCl₂, 400 mM KCl) with 28 nM [³H]-fMet-tRNA, 1 mM puromycism at varying concentrations of LC. Reactions were initiated by addition of 25 μl methanol and incubated on ice for 30 min. Reactions were stopped and analyzed as described (Maguire et al., 2005, Mol Cell 20, 427-435). In the ribosome-based puromycin assay, 70S ribosomes of S. aureus (at a final concentration of 200 nM) were preincubated for 15 min at 37° C. in 50 μl of polyamine buffer (20 mM HEPES-KOH, pH 7.6, 6 mM Mg-acetate, 150 mM NH₄Cl, 4 mM β-mercaptoethanol, 2 mM spermidine, 0.05 mM spermine) with 600 nM mRNA AAGGAGAUAAACAAUGGGU and 28 nM [³H]-fMet-tRNA. After addition of varying concentrations of LC, puromycin was added to the final concentration of 0.5 mM and reactions were incubated for 15 m in at 37° C. Reactions were stopped and processed as in Maguire et al ibid.

### RNA Probing

RNA probing was carried out essentially as described (16, 46) using D. radiodurans 50S ribosomal subunits at 200 nM concentration and antibiotics at the following concentrations: LC—50 μM, LM—500 μM, ERY—50 μM. Prior to addition of the modifying reagents, ribosomal subunits were pre-incubated with the drug 10 min at 37° C. and then 10 min at 20° C.

### Cell-Free Translation System Used for Detecting Synergism In Vitro

Inhibition of cell-free protein synthesis by LC and LM was measured using the E. coli cell-free transcription-translation assay. Cell free extract was prepared from Escherichia coli (Strain BL21-DE3) in a manner similar to that previously reported (Kim, T. W. et al., 2006, J Biotechnol 126, 554-561). An aqueous solution containing amino acid mix (1.3 mM for each amino acid), magnesium acetate (20 mM), NTP mix (0.9 mM), ATP (0.4 mM), potassium glutamate (150 mM), E. coli tRNA mixture (0.17 mg/mL), DTT (1.8 mM), folinic acid (35 μg/mL), cAMP (0.65 mM), NH₄OAc (28 mM), creatine phosphate (80 mM), HEPES (pH=7.5, 37 ° C., 140 mM), 9.5% w/v PEG-8000, tyrosine (0.4 mg/mL), creatine kinase (0.35 mg/mL) and S12 cell free extract (12% v/v) was prepared. PIVEX.6D plasmid encoding wild-type GFP (1 ng/μL) was added to this solution commencing the transcription-translation reaction.

This solution was immediately added to a 96 well plate containing serial dilutions of lankamycin and lankacidin. After incubation for 1 hour at 37° C., 50 μL of erythromyin (8 μM) was added to each well to completely stop the translation reaction. The fluorescence data were collected on a TECAN SpectraFluor Plus™ 96-well plate reader (λ_{excite}=485 nm, λₑₘᵢₜ=535 nm). The data were fitted using the least-squared regression analysis package in Igor Pro™.

A control experiment was performed in the absence of the antibiotics. This experiment was also used for the determination of the time window required for maximum GFP production before the reaction was quenched.

### Pharmacology

The antibiotics and combinations of the present invention can be administered to an organism per se, or in a pharmaceutical composition where it is mixed with suitable carriers or excipients.

As used herein, a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Hereinafter, the phrases “physiologically acceptable carrier” and “pharmaceutically acceptable carrier,” which may be used interchangeably, refer to a carrier or a diluent that does not cause significant irritation to an organism and does not abrogate the biological activity and properties of the administered compound. An adjuvant is included under these phrases.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

Techniques for formulation and administration of drugs may be found in the latest edition of “Remington's Pharmaceutical Sciences,” Mack Publishing Co., Easton, Pa., which is herein fully incorporated by reference.

Suitable routes of administration may, for example, include oral, rectal, transmucosal, especially transnasal, intestinal, or parenteral delivery, including intramuscular, subcutaneous, and intramedullary injections, as well as intrathecal, direct intraventricular, intravenous, inrtaperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping, or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations that can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include push-fit capsules made of gelatin, as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane, or carbon dioxide. In the case of a pressurized aerosol, the dosage may be determined by providing a valve to deliver a metered amount. Capsules and cartridges of, for example, gelatin for use in a dispenser may be formulated containing a powder mix of the compound and a suitable powder base, such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with, optionally, an added preservative. The compositions may be suspensions, solutions, or emulsions in oily or aqueous vehicles, and may contain formulatory agents such as suspending, stabilizing, and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acid esters such as ethyl oleate, triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

Alternatively, the active ingredient may be in powder form for constitution with a suitable vehicle, e.g., a sterile, pyrogen-free, water-based solution, before use.

The pharmaceutical composition of the present invention may also be formulated in rectal compositions such as suppositories or retention enemas, using, for example, conventional suppository bases such as cocoa butter or other glycerides.

Pharmaceutical compositions suitable for use in the context of the present invention include compositions wherein the active ingredients are contained in an amount effective to achieve the intended purpose. More specifically, a “therapeutically effective amount” means an amount of active ingredients (e.g., a nucleic acid construct) effective to prevent, alleviate, or ameliorate symptoms of a disorder (e.g., ischemia) or prolong the survival of the subject being treated.

Determination of a therapeutically effective amount is well within the capability of those skilled in the art, especially in light of the detailed disclosure provided herein.

For any preparation used in the methods of the invention, the dosage or the therapeutically effective amount can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition. (e.g., Fingl, E. et al. 1975, “The Pharmacological Basis of Therapeutics,” Ch. 1, p. 1).

Dosage amount and administration intervals may be adjusted individually to provide sufficient plasma or brain levels of the active ingredient to induce or suppress the biological effect (i.e., minimally effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks, or until cure is effected or diminution of the disease state is achieved .

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA-approved kit, which may contain one or more unit dosage forms containing the active ingredient. The pack may, for example, comprise metal or plastic foil, such as a blister pack. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising a preparation of the invention formulated in a pharmaceutically acceptable carrier may also be prepared, placed in an appropriate container, and labeled for treatment of an indicated condition, as further detailed above.

### Chemical Derivatives and Conjugates

Derivatives of the molecules of the present invention are defined by at least one replacement, addition or deletion of an atom in the parent molecule by another atom, providing that the derived molecule retains at least some of the properties of the parent molecule and/or possess at least one improved property.

Derivatives of the molecules of the present invention are produces based on X-ray crystallographic analysis of the ribosomal complexes of lankacidin and lankamycin, alone or in conjunction with each other. The analysis revealed that the binding mode and position of lankamycin (the macrolide component) was extremely closely related to the archetypical macrolide, erythromycin. However, lankamycin lacks a critical element, common to most ribosomal interfering antibiotics; a positive charge. Erythromycin, bearing a positively charged desoamine pendant sugar moiety, forms a salt bridge with the rRNA phosphate backbone. Lankamycin in the same position has a neutrally charged chalcose sugar, leading to substantially lower ribosomal inhibition.

Desoamine derivatives of lankamycin were designed and are tested, both in their ability to interact with positive synergy with lankacidin and for their antibacterial effect. The structures of D50S-LC, D50S-LM and D50S-LM/LC supported by detailed chemical footprinting of 23S rRNA, disclosed herein for the first time, are used to develop more potent 50S ribosomal interfering antibiotics that simultaneously bind two crucial sites in the ribosome. These improved antibiotic molecules may comprise for example: physical enlargement of one or both of the synergistic components, covalent linkage of the two molecules, modification of the macrolide component (for example LM) to further strengthen its binding (for example by adding a positive charge moiety).

### Synthetic and Semi-Synthetic Methods for Producing Antibiotics

Unmodified antibiotics, used for example for synergistic combinations, may be produced by fermentation processes known in the art. Synthetic and semi-synthetic methods know in the art for production of antibiotic molecules and derivatives thereof can be also used to produce the molecules of the present invention. For example, U.S. Pat. No. 4,290,947 discloses a chiral synthesis of thienamycin starting from L-aspartic acid. EP 0080763 discloses chemical process for the synthesis of macrolide antibiotics. US 20060074032 describes synthesis and separation of optically active isomers of erythromycin. U.S. Pat. No. 7,339,066 discloses and claims intermediates for the synthesis of polypropionate antibiotics. WO 2004/062558 discloses process for enzymatic synthesis of beta-lactam antibiotics. U.S. Pat. No. 6,403,776 discloses and claims synthesis of carbamate ketolide antibiotics. US 20090312530 discloses processes for the convergent synthesis of calicheamicin derivatives. The contents of the aforementioned references are hereby incorporated by reference in their entirety.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. to 36. (canceled)

37. A pharmaceutical composition comprising two antibiotic molecules capable of binding simultaneously to neighboring sites of a ribosomal large subunit, wherein one antibiotic is capable binding to the peptidyl transferase center site of the ribosomal large subunit and the other antibiotic is capable of binding to the nascent peptide exit tunnel site of the ribosomal large subunit, wherein at least one of the antibiotic molecules is selected from the group consisting of Lankacidin C, Lankamycin, Virginiamycin M, Virginiamycin S, Pleuromutilin, Chloramphenicol, Methylmycin and Clindamycin, and wherein the two antibiotic molecules provide a synergistic anti-bacterial effect.

38. The pharmaceutical composition according to claim 37, wherein one of the antibiotic molecules is a Lankamycin derivative.

39. The pharmaceutical composition according to claim 37, wherein the antibiotic combination is selected from the group consisting of Lankacidin C and Quinupristin; Lankacidin C and Virginiamycin S; Lankamycin and Dalfopristin; Lankamycin and Virginiamycin M; Dalfopristin and Virginiamycin S; and Quinupristin and Virginiamycin M.

40. The pharmaceutical composition according to claim 37, wherein one of the antibiotic molecules is Methylmycin.

41. The pharmaceutical composition according to claim 38, wherein one of the antibiotic molecules is a Lankamycin derivative represented by the structure of Formula I: 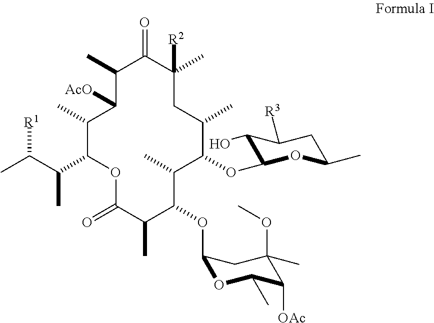
wherein:
R¹ is OH, R² is H, and R³ is OMe; or
R¹ is H, R² is OH, and R³ is OMe; or
R¹ is H, R² is H, and R³ is OMe; or
R¹ is OH, R₂ is OH, and R³ is selected from the group consisting of: NMe₂, NHMe, and NH₂; or
R¹ is an amine-containing group, R² is OH, and R³ is OMe or NMe₂; or
R¹ is selected from the group consisting of: amine, N-methyl amine, N-dimethyl amine, N-ethyl amine, R² is OH, and R³ is OMe or NMe₂.

42. The pharmaceutical composition according to claim 38 wherein the Lankamycin derivative is 3-O-arcanosyl Lankamycin represented by the structure of Formula II:

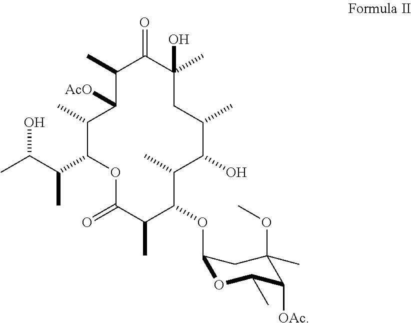

43. The pharmaceutical composition according to claim 38, wherein the antibiotic combination is a Lankamycin derivative and Lankacidin C.

44. The pharmaceutical composition according to claim 37, wherein the antibiotic combination is Lankamycin or a derivative thereof and a type A Streptogramin such as Dalfopristin.

45. The pharmaceutical composition according to claim 3, wherein the antibiotic combination is Lankacidin C and a type B Streptogramin such as Quinupristin.

46. The pharmaceutical composition according to claim 37, wherein the antibiotic which is capable of binding to the peptidyl transferase center site of the ribosomal large subunit is selected from the group consisting of Lankacidin C, Virginiamycin M, pleuromutilin, Chloramphenicol, Methylmycin and Clindamycin.

47. The pharmaceutical composition according to claim 37, wherein the antiobiotic which is capable of binding to the nascent peptide exit tunnel site of the ribosomal large subunit is selected from the group consisting of Lankamycin and Virginiamycin S.

48. The pharmaceutical composition according to claim 37, wherein the antibiotic combination is Lankacidin C and at least one antibiotic selected from the group consisting of Lankamycin, Quinupristin, and Virginiamycin S.

49. The pharmaceutical composition according to claim 37, wherein the antibiotic combination is Lankamycin M or a derivative thereof, and at least one antibiotic selected from the group consisting of Lankacidin C, Dalfopristin and Virginiamycin.

50. A chimeric molecule comprising at least a portion of a first antibiotic molecule capable of binding to the peptidyl transferase center site of the ribosomal large subunit and at least a portion of a second antibiotic molecule capable of binding simultaneously to the nascent peptide exit tunnel site of the same ribosomal large subunit wherein the first and the second antibiotic molecules together provide a synergistic anti-bacterial effect.

51. A method for prevention and treatment of bacterial infection comprising administering to a subject in need thereof, a pharmaceutical composition according to claim 37.

52. The method according to claim 51 wherein bacterial infection involves gram-positive bacteria.

53. A Lankamycin derivative presented by Formula I: 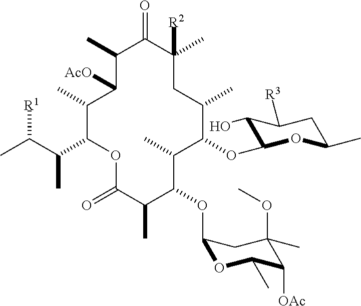
wherein:
R¹ is OH, R² is OH, and R³ is selected from the group consisting of: NMe₂, NHMe, and NH₂; or
wherein R¹ is an amine-containing group, R² is OH, and R³ is OMe or NMe₂; or
wherein R¹ is selected from the group consisting of: amine, N-methyl amine, N-dimethyl amine, N-ethyl amine, R² is OH, and R³ is OMe or NMe₂.

54. A pharmaceutical composition comprising at least one Lankamycin derivative according to claim 53.

55. A method for prevention and treatment of bacterial infection comprising administering to a subject in need thereof, a Lankamycin derivative according to claim 53 alone or in the form of a pharmaceutical composition.

56. The method according to claim 58 wherein bacterial infection involves gram-positive bacteria.
